(19) United States
(12) Patent Application Publication
(10) Pub. No.: US 2019/0350826 A1
(43) Pub. Date: Nov. 21, 2019

(54) FRAGRANCE AND FLAVOR MATERIALS

(71) Applicant: TAKASAGO INTERNATIONAL CORPORATION, Tokyo (JP)

(72) Inventors: Maureen Blandino, Dumont, NJ (US); Michael E. Lankin, High Bridge, NJ (US)

(73) Assignee: TAKASAGO INTERNATIONAL CORPORATION, Tokyo (JP)

(21) Appl. No.: 16/466,080

(22) PCT Filed: Dec. 22, 2017
(86) PCT No.: PCT/US2017/068361

§ 371 Date: Jun. 3, 2019

Related U.S. Application Data
(60) Provisional application No. 62/438,385, filed on Dec. 22, 2016.

(51) Int. Cl. A61K 8/35; A23L 27/20; A61Q 13/00

(52) U.S. Cl. CPC A61K 8/35; A61Q 13/00; A23L 27/2026

(57) ABSTRACT

The present disclosure is directed to the synthesis and application of an ethyl linalool derivative having unique and desired flavor and/or fragrant characteristics. Specifically, the compound is represented by Formula (I). The compound of Formula (I) can be employed alone or incorporated as fragrance or flavor ingredients in fragrance or flavor compositions. The present disclosure is also directed to consumer products comprising such derivatives and/or fragrance or flavor compositions.

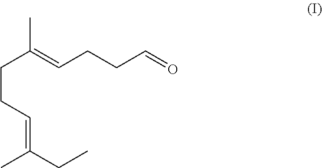

### CROSS-REFERENCE TO RELATED APPLICATIONS

The present application claims priority to U.S. Provisional Application No. 62/438,385, filed on Dec. 22, 2016, the content of which is incorporated herein by reference in its entirety.

### FIELD OF THE INVENTION

The present application relates to a compound useful as a fragrance or flavor component in fragrance or flavor compositions.

### BACKGROUND OF THE INVENTION

There is a continuing interest in the preparation of synthetic fragrance and flavor components and their use in consumer products. One strategy to prepare such compounds is to apply a known synthetic chemical reaction to readily available substrates. The Claisen Rearrangement is a reaction known to one skilled in the art of organic synthesis. When an allylic alcohol is converted to a vinyl ether and treated with acid and heat, the vinyl ether rearranges to a 4-alkenal. There are several Claisen products known for their use in the flavor and fragrance industry (Nowicki, Molecules 2000, 1033-1050).

There remains a need and demand for unique fragrance and flavor compounds. There is also a need for fragrance and flavor compositions with pleasing and consumer preferred odor and taste profiles for use in multiple consumer products.

### SUMMARY OF THE INVENTION

The present disclosure is directed to the synthesis and application of an ethyl linalool derivative having unique and desired flavor and/or fragrant characteristics.

The compound of the present disclosure can be employed alone or incorporated into fragrance or flavor compositions.

In certain embodiments, the presently disclosed subject matter provides a fragrance or flavor composition for addition to a consumer product comprising a compound represented by Formula (I),

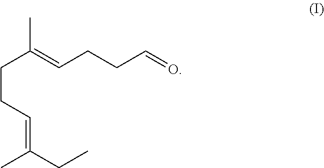

In certain embodiments, the compounds include constitutional isomers, enantiomers, stereoisomers, and racemic mixtures of said compounds listed herein.

Another aspect of the present disclosure provides a fragrance or flavor composition for addition to a consumer product comprising one or more compounds of Formula (I) in an amount effective to impart a fragrance or flavor to the consumer product.

### DETAILED DESCRIPTION

### Definitions

The terms used in this specification generally have their ordinary meanings in the art, within the context of this disclosure and in the specific context where each term is used. Certain terms are discussed below, or elsewhere in the specification, to provide additional guidance to a person of ordinary skill in the art describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” a plurality, and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “enantiomers” refers to a pair of stereoisomers that are non-superimposable mirror images of each other. A 1:1 mixture of a pair of enantiomers is a “racemic” mixture. The term is used to designate a racemic mixture where appropriate.

As used herein, the term “diastereoisomers” refers to stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon may be specified by either R or S. Resolved compounds whose absolute configuration is unknown can be designated (+) or (−) depending on the direction (dextro or levorotatory) in which they rotate plane polarized light at the wavelength of the sodium D line. The compounds of the presently disclosed subject matter contain one or more asymmetric centers and may thus give rise to enantiomers, diastereomers, and other stereoisomeric forms that may be defined, in terms of absolute stereochemistry, as (R)- or (S)-. The presently disclosed subject matter is meant to include all such possible isomers, including racemic mixtures, optically pure forms, and intermediate mixtures. Optically active (R)- and (S)-isomers may be prepared using chiral synthons or chiral reagents, or resolved using conventional techniques. If the compound contains a double bond, the substituent may be E or Z configuration. All tautomeric forms are also intended to be included.

As used herein, the term “isomers” refers to different compounds that have the same molecular formula but differ in arrangement and configuration of the atoms. Also as used herein, the term “stereoisomer” refers to any of the various stereo isomeric configurations which may exist for a given compound of the presently disclosed subject matter and includes geometric isomers. It is understood that a substituent may be attached at a chiral center of a carbon atom. Therefore, the presently disclosed subject matter includes enantiomers, diastereomers, or racemates of the compound. Also as used herein, the terms “constitutional isomers” refers to different compounds which have the same numbers of, and types of, atoms but the atoms are connected differently.

As used herein, the term “fragrance composition” refers to a mixture comprising one or more fragrance components, in any of their forms, and one or more solvents or perfuming co-ingredients. As known in the art, a fragrance composition contains one or more fragrance components (e.g., perfuming co-ingredients) in order to impart an olfactory note to the composition (e.g., a household cleaner, perfume, or other consumer product) to which it is added. In one embodiment, the fragrance composition contains two or more fragrance components which, collectively and in combination with the solvent to which they are added, impart an intended olfactory note (e.g., a hedonically pleasing “tropical” note) to a human in close proximity to the fragrance composition.

In general terms, perfuming co-ingredients belong to chemical classes as varied as alcohols, aldehydes, ketones, esters, ethers, acetates, nitriles, terpene hydrocarbons, nitrogenous or sulphurous heterocyclic compounds and essential oils of natural or synthetic origin, and are known to perfumists of ordinary skill in the art. Many of these ingredients are listed in reference texts such as S. Arctander, Perfume and Flavor Chemicals, 1969, Montclair, N.J., USA or any of its more recent versions, each of which are hereby incorporated by reference.

As used herein, the term “flavor composition” refers to a composition that contains one or more compounds (e.g., co-ingredients) that provide a desired taste when combined with a solvent that is suitable for oral administration and oral consumption. Examples of flavoring co-ingredients that are generally included in a flavor composition are listed in S. Arctander, Perfume and Flavor Chemicals, 1969, Montclair, N.J., USA. The skilled person in the art of flavors is able to select them on the basis of its general knowledge and according to the nature of the product to be flavored and the desired taste.

As used herein, the phrase “consumer product” or “end product” refers to composition that is in a form ready for use by the consumer for the marketed indication. A solvent suitable for use in a consumer product is a solvent that, when combined with other components of the end product, will not render the consumer product unfit for its intended consumer use.

### Ethyl Linalool Derivative

The present disclosure is directed to the synthesis and application of an ethyl linalool derivative having unique and desired flavor and/or fragrant characteristics.

The structure of the compound of Formula (I) is illustrated below:

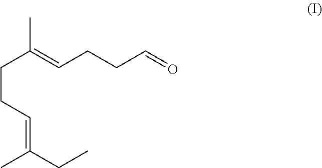

The compound of the present disclosure can be prepared synthetically. In certain embodiments, the compound of the present disclosure is prepared by a Claisen Rearrangement of ethyl linalool.

The Claisen Rearrangement is a reaction known to those skilled in the art of organic synthesis. When an allylic alcohol is converted to a vinyl ether and treated with acid and heat, the vinyl ether rearranges to a 4-alkenal. There are several Claisen products already known for their use in the flavor and fragrance industry (Nowicki, Molecules 2000, 1033-1050).

In certain embodiments, a compound of Formula (I) can be prepared by applying the Claisen Rearrangement to ethyl linalool.

For the Claisen Rearrangement, the allylic alcohol can be treated with various vinyl reagents to generate the vinyl ether intermediate, including but not limited to ethyl vinyl ether, butyl vinyl ether, and isopropenyl methyl ether. The Claisen

Rearrangement can be catalyzed by various acids, including but not limited to mercury diacetate, p-toluenesulfphonic acid, phosphoric acid, phenyl phosphonic acid, propionic acid, palladium diacetate, palladium ditriflate, and 1,10-phenanthroline complexes of palladium diacetate and palladium ditriflate. Sometimes a base is added, such as pyridine, triethyl amine, or triethanolamine. The Claisen Rearrangement can be performed neat or in a solvent such as toluene or xylenes. See J. Chem. Soc. Perkin Trans. I 1985, page 817, GB1281813, J. Org. Chem. 2007, 72, 4250-4253 and J. Org. Chem. 1976, pages 3497-3505.

### Fragrance or Flavor Compositions

As noted above, the compound of Formula (I) can be included in a fragrance or flavor composition.

### Fragrance Compositions

In certain embodiments, the compound can be provided in a fragrance composition. Certain embodiments of the presently disclosed subject matter provide a method to modify, enhance or improve the odor properties of a fragrance composition by adding to the composition an effective quantity of the compound of Formula (I).

The compound of the present disclosure is particularly valuable as being capable of imparting mandarin and citrus rind notes to a fragrance composition. For fragrance applications, typical concentrations of the compounds of Formulas (I) range from about 0.001% to about 20% by weight, preferably from about 0.01% to about 10% by weight, more preferably from about 0.1% to about 5%, more preferably from about 1% to about 3%, based on the total weight of the composition into which the compound is incorporated.

In other embodiments of fragrance compositions, the concentrations of the compounds of Formula (I) range from about 0.01% to about 8% by weight, or from about 0.01% to about 5% by weight, or from about 0.01% to about 4%, or from about 0.01% to about 3%, or from about 0.01% to about 2%, from about 0.01% to about 1%, or from about 0.1% to about 1%, or from about 0.2% to about 1%, based on the total weight of the composition into which the fragrance compound is incorporated. Those skilled in the art are able to employ the desired level of the compound of the disclosed subject matter to provide the desired fragrance/flavor and intensity. In general, the compound of the present disclosure can be used in relatively small amounts, typically via significant dilutions due to their high-impact, diffusive properties.

The compounds of the presently disclosed subject matter can be combined with one or more fragrance accords or compounds from various fragrance categories including but not limited to one or more aldehydic compound(s), one or more animalic compound(s), one or more balsamic compound(s), one or more citrus compound(s), one or more floral compound(s), one or more fruity compound(s), one or more gourmand compound(s), one or more green compound(s) one or more herbaceous compound(s) one or more marine compound(s), one or more mossy compound(s), one or more musk compound(s), one or more piney compound(s), one or more powdery compound(s), one or more spicy compound(s) and/or one or more woody compound(s), and combinations thereof.

Non-limiting examples of suitable aldehydic compounds include acetaldehyde C-8, acetaclehdye C-9, acetaldehyde C-10, adoxal, aldehyde C-8, aldehyde C-9, aldehyde C-10, aldehyde C-11, aldehyde C-12, aldehyde C-12 lauric, aldehyde C-12 MNA, aldehyde supra, cyclomyral trans-2-decenal, trans-4-decenal cis-4-decenal, 9-decenal, myrac aldehyde, precyclemone B, trans-2-decenal, undecylenic aldehyde, VERNALDEHYDE®, and combinations thereof.

Non-limiting examples of an animalic compound are AMBRETONE®, ambrettolide, ambrinol, ALDRON®, civet, p-cresol, indole, skatole, and combinations thereof.

Non-limiting examples of a balsamic compound are benzy salicylate, cylohexyl salicylate, isopropoxy ethyl salicylate, phenethyl salicylate, styrax oil, and combinations thereof.

Non-limiting examples of a citrus compound are delta-3-carene, citral, citronellal, L-citronellol, decanal, limonene, myrcenol, nootkatone, sinensal, bergamot oil, grapefruit oil, lemon oil, lime oil, orange oil, tridecene-2-nitrile, and/or yuzu core base.

Non-limiting examples of a floral compound are acetanisole, alpha amyl cinnamaldehyde, anisyl acetate, anisic aldehyde, benzyl acetate, bourgeonal, butyl acetate, 1-citrol, cyclamen aldehyde, cyclohexyl lactone, delta-damascone, 9-decen-1-ol, dimethyl benzyl carbinol, farnesal, 1-dihydrofarnesal, 1-farnesal, farnesol, FLORHYDRAL®, floralozone, FLOROL®, geraniol, gernayl acetate, piperonal, HEDIONE®, heliobouquet, hexyl cinnamaldehyde, hexyl salicylate, indole, alpha-ionone, beta-ionone, isopropoxy ethyl salicylate, JASMODIONE®, cis-jasmone, KOVANOL®, LAURINAL®, lilial, linalool, linalyl acetate, LOREXAN®, magnolan, MAYOL®, methyl dihydrojasomante, gamma-methyl ionone, methoxymelonal, nerol, nerolione, neryl acetate, 2-pentyl cyclopentanone, PEONILE®, phenoxanol, phenoxy ethyl isobutyrate, phenylacetaldehyde, phenyl ethyl alcohol, rose oxide, rosephenone, SUZARAL®, terpineol, undecavertol, VELOUTONE®, yara yara, geranium oil, lavender oil, rose oil, ylang oil, and combinations thereof.

Non-limiting examples of a fruity compound are aldehyde C-16, allyl caproate, allyl cyclohexyl proprionate, allyl heptanoate, amyl acetate, benzaldehyde, CASSIS®, L-citronellyl acetate, L-citronellyl nitrile, CYCLACET®, CYCLAPROP®, damascenone, beta-decalactone, gamma-decalactone, diethyl malonate, dimethyl benzyl carbinol acetate, dimethyl phenyl ethyl carbinol, dimethyl sulfide, y-dodecalactone, ethyl acetate, ethyl butyrate, ethyl caproate, ethyl decadienotate, ethyl-2-methylbutyrate, ethyl acetoacetate, ethyl propionate, FLOROL®, FRUITATE®, hexyl acetate, hexyl isobutyrate, isoamyl acetate, jasmolactone, manzanate, melonal, methyl heptyl ketone, gamma-nonalactone, gamma-octalactone, phenyl ethyl isobutyrate, raspberry ketone, RINGONOL®, THESARON®, tolyl aldehyde, γ-undecalactone, vanoris, verdox, and combinations thereof.

Non-limiting examples of a gourmand compound are butyl butyryl lactate, caprylic acid, coumarin, ethyl fraison, ethyl vanillin, ethyl maltol (e.g., Veltol Plus), filbertone, FURANEOL®, guaiacol, maple furanone, 2-acetyl pyrazine, 2,5-dimethyl pyrazine, vanillin, and combinations thereof.

Non-limiting examples of a green compound are allyl amyl glycolate, DYNASCONE®, galbanolene, trans-2-hexenal, cis-3-hexenol, hexen-1-ol, cis-3-hexenyl acetate, cis-3-hexenyl butyrate, cis-3-hexenyl formate, cis-3-hexenyl salicyclate, liffarome, 2-methoxy-2-methylheptane, methyl octine carbonate, neofolione, 2,6-nonadienal, OXANE®, SCENTENAL®, STEMONE®, styrallyl acetate, TRIPLAL®, undecavertol, violet methyl carbonate (e.g., violet T), vionil, violet leaf extract, and combinations thereof.

Non-limiting examples of a herbaceous compound are bamboo ketone, canthoxal, carvacrol, clary sage natural oil, DIMETOL®, natural basil oil, natural cedar leaf oil, and combinations thereof.

Non-limiting examples of a marine compound are Calone®1951, floralozone, MARENIL®, myrac aldehyde, ultrazure, and combinations thereof.

Non-limiting examples of a mossy compound are hinokitiol, isobutyl quinolone, isopropyl quinolone and/or oakmossTM #1, and combinations thereof.

Non-limiting examples of a musk compound are ambrettolide, AMBRETONE®, AMBROXAN®, EXALTOLIDE®, GALAXOLIDE®, HABANOLIDE®, HELVETOLIDE®, (1′R)-3-methyl-5-(2,2,3 -trimethylcyclopentan-1-yl)-2-pentanone, MUSCENONE®, musk T, L-muscone, TONALID®, and combinations thereof.

Non-limiting examples of a piney compound are 1-borneol, 1-bornyl acetate,camphene, dihydroterpineol, β-pinene, and combinations thereof.

Non-limiting examples of a powdery compound are heliotropine and/or whiskey lactone (methyl octalactone).

Non-limiting examples of a spicy compound are delta-caryophellene, cinnamaldehyde, cuminaldehyde, eugenol, isoeugenol, perilla aldehyde, cardamom oil, clove oil, ginger extract, black pepper extract and combinations thereof.

Non-limiting examples of a woody and/or amber compound are amber core, amber extreme, ambretol, AMBROCENIDE®, AMBROXAN®, bacdanol, boisambrene forte, cashmeran, cedramber, cedanol, EBANOL®, HINDINOL®, hinokitiol, dH-ionone beta, JAVANOL®, karanal, OKOUMAL®, ORBITONE®, patchouly oil, sandalwood, and combinations thereof.

The compound of Formula (I) can be used from about 0.01% to about 10% in a fragrance. More specifically, in a bar soap, the compound of Formula (I), where R is hydrogen, can be used from about 0.2% to about 2% in a fragrance, which is then used at up to about 1% in a bar soap. Additionally, in an all-purpose cleaner, the compound of Formula (I), where R is hydrogen, can be used from about 0.2% to about 1%, but most often used at about 0.2% in a fragrance, which is then used at up to about 0.35% in an all-purpose cleaner. Also, in a shampoo, the compound of Formula (I), where R is hydrogen, can be used from about 0.2% to about 1%, but most often used at about 0.2% in a fragrance, which is then used at up to about 0.85% in a shampoo. The compound of Formula (I), where R is hydrogen, can also be use in a fine fragrance from about 0.01% to about 0.5%, but most often used at about 0.1% in a fragrance, which is then used at up to about 10% in a fine fragrance.

The fragrance profile of the compound of Formula (I) is in the same area as other unsaturated aldehydes of similar molecular weight or with a similar number of carbon atoms (for example, trans 2-dodecenal, floral super, trans 2-tridecenal). The compound of Formula (I) can be used in conjunction with many types of fragrance compounds but especially florals and citrus materials to add sparkle, volume and floralcy.

In certain embodiment, the compounds of the presently disclosed subject matter can be combined with one or more fragrance compounds from various fragrance categories including but not limited to amber, citrus, floral, fruity, green, musky, spicy, and woody. The amounts of the fragrance compounds can vary depending on the intended resulting fragrance composition, but can range from about 0.1 parts per thousand to about 800 parts per thousand, or from about 1 part per thousand to about 500 parts per thousand.

Such compositions can contain or consist of at least one ingredient selected from a group consisting of a fragrance carrier and a fragrance base. Such compositions can also consist of at least one fragrance adjuvant.

Fragrance carriers can be a liquid or a solid and typically do not significantly alter the olfactory properties of the fragrance ingredients. Some non-limiting examples of fragrance carriers include an emulsifying system, encapsulating materials, natural or modified starches, polymers, gums, pectins, gelatinous or porous cellular materials, waxes, and solvents which are typically employed in fragrance applications.

Fragrance base refers to any composition comprising at least one fragrance co-ingredient. In general, these co-ingredients belong to chemical classes such as, but not limited to: alcohols, aldehydes, ketones, esters, ethers, acetals, oximes, acetates, nitriles, terpenes, saturated and unsaturated hydrocarbons, and essential oils of natural or synthetic origins.

The fragrance compositions according to the disclosed subject matter can be in the form of a simple mixture of the various co-ingredients and solvents, or also in the form of a biphasic system such as an emulsion or microemulsion. Such systems are well-known to persons skilled in the art.

Nonlimiting examples of such solvents used in perfumery are known in the art and include but are not limited to: dipropyleneglycol, diethyl phthalate, isopropyl myristate, benzyl benzoate, 2-(2-ethoxy)-1-ethanol, ethyl citrate, ethanol, water/ethanol mixtures, limonene or other terpenes, isoparaffins such as those known under the trademark Isopar® (ExxonMobil Chemicals, Houston, Tex.), and glycol ethers and glycol ether esters such as those known under the trademark Dowanol® (Dow Chemical Company, Midland Mich.).

Suitable consumer products that can include a fragrance compound or fragrance composition of the presently disclosed subject matter include but are not limited to: 1) air care products, e.g., candles, air fresheners; 2) fine fragrances; 3) personal care products, e.g., soaps, deodorants, adult and baby shampoos, conditioners, shower gels, shaving lotions, infant and toddler care wipes; 4) cosmetics, e.g., lotions, creams and ointments for the skin, color cosmetics; 5) fabric and home care products, e.g., laundry detergents, fabric softeners and conditioners, rinse additives; dish detergents, household and institutional cleansers/cleaning agents; 6) pharmaceutical and over-the-counter (OTC) products, e.g., medicated soaps, medicated shampoos, medicated skin care products; and 7) insect repellents or insecticides. Depending on the solvents that can be present in some end products, it can be necessary to protect the compounds from premature degradation, for example by encapsulation or with a stabilizer, or other methods well-known to those of ordinary skill in the art.

The above-listing of end products is non-limiting. The compositions of the presently disclosed subject matter can be included in a number of additional products. Non-limiting examples of fine fragrance products include eau de perfume, eau de toilet, eau de cologne, and the like.

Non-limiting examples of cosmetics include skin-care cosmetics, face washing creams, varnishing creams, cleansing creams, cold creams, massage creams and oils, milky lotions, skin toning lotion, makeup remover, makeup cosmetics, foundations, face powders, pressed powders, talcum powders, lip sticks, lip creams, cheek powders, eyeliners, mascara, eye shadows, eyebrow pencils, eye packs, nail enamels, nail enamel removers, suntan products, sunscreen products, and the like.

Non-limiting examples of hair care products include pomades, rinses, brilliantines, setting lotions, hair sticks, hair solids, hair oils, hair treatments, hair creams, hair tonics, hair liquids, hair sprays, hair restorers, hair dyes, permanent wave lotions, and the like.

Non-limiting examples of personal care products include antiperspirants, after-shave lotions and gels, bath soaps, perfumed soaps, transparent soaps, synthetic soaps, body shampoos, hand soaps, bath salts, bath tablets, bath liquids, foam and bubble baths, bath oils, bath perfumes, bath capsules, milk baths, bath gels, bath cubes, and the like.

Non-limiting examples of laundry care products include heavy duty detergents for clothes, light duty detergents for clothes, liquid detergents, laundering soaps, compact detergents, powder soaps, softening finishing agents, and softeners.

Non-limiting examples of home care products include furniture care products, car care products and the like; insect repellent, insecticides, and the like

### Flavor Compositions

In certain embodiments, the compound of Formula (I) can be provided in a flavor composition. Certain embodiments of the presently disclosed subject matter provide a method to modify, enhance, or improve the taste properties of a flavor composition by adding to the composition an effective quantity of the compound of Formula (I).

The compound of the present disclosure is particularly valuable as being capable of imparting mandarin and citrus rind notes to flavor ingredients, and may be particularly suitable as a replacement for mandarin aldehyde, octanal, nonanal, or decanal in certain flavor compositions and at various levels. For flavor applications, concentrations of the compound of Formula (I) are based on the total weight of the composition into which the flavor compound is incorporated. For flavor applications, typical concentrations of the compound of Formula (I) range from about 0.001% to about 20% by weight, preferably from about 0.01% to about 10% by weight, more preferably from about 0.1% to about 5%, more preferably from about 1% to about 3%, based on the total weight of the composition into which the compound is incorporated. Those skilled in the art are able to employ the desired level of said compound to provide the desired flavor and intensity. Much higher concentrations can be employed when the compound is used in concentrated flavors and flavor compositions.

As used herein, organoleptic effective quantity will be defined as the amount of said compound in a flavor composition in which the individual component will contribute its characteristic flavor properties. However, the organoleptic effect or taste profile of the flavor composition will be the sum of the effects of all flavor ingredients present. Therefore, the compounds embodied in the presently disclosed subject matter can be employed in more complex flavor compositions comprising one or more other flavor ingredients, or other flavor compositions, to modify the overall taste characteristics of the flavor composition via their own organoleptic properties or through enhancing or complimenting the contributions of the other flavor ingredient(s) present within the said composition. Such other flavor ingredients or flavor compositions include, for example, natural or synthetic flavors, i.e., fruit flavors (e.g., lemon, lime, orange, grapefruit; cherry, strawberry, raspberry, cranberry; apple, grape, pineapple, banana, tomato); natural or synthetic botanical flavors (e.g., tea flavors, coffee flavors, hazelnut, almond, pecan or other nut flavors; vanilla flavors), and flavor compositions with complex flavor profiles (e.g., cola flavors or imagined flavors, such as “birthday cake” or “ice cream sundae”). The quantity of the presently disclosed compound in such more complex flavor compositions will vary widely depending on the presence of other ingredients present, their relative amounts, the desired taste profile, and the nature of the consumer product in which the flavor composition will be utilized.

The flavor carrier can be a liquid or a solid, and typically does not significantly alter the olfactory or organoleptic properties of the flavor ingredients, respectively. Some non-limiting examples of flavor carriers include an emulsifying system, encapsulating materials, natural or modified starches, polymers, pectins, proteins, polysaccharides, gums and solvents which are typically employed in flavor applications.

The flavor compositions according to the disclosed subject matter can be in the form of a simple mixture of the various co-ingredients, adjuvants, and solvents, or also in the form of a biphasic system such as an emulsion or microemulsion. Such systems are well-known to persons skilled in the art.

The flavor compositions of the presently disclosed subject matter can further comprise one or more support materials. By way of non-limiting example, support materials can include diluents, e.g., ethanol, purified water, glycerol; solvents; carriers, e.g., propylene glycol, triacetin; preservatives, e.g., sulfites, sodium nitrite, propionic acid, sorbic acid, benzoic acid, disodium ethylenediaminetetraacetic acid

(EDTA); flavoring agents, e.g., alcohols, esters, aldehydes; ketones, lactones, phenols, flavor enhancers, e.g., monosodium glutamate (MSG), monopotassium glutamate, calcium diglutamate (CDG), guanosine monophosphate disodium guanylate, sodium guanylate, inosinic acid and its salts, L-leucine; antioxidants, e.g, ascorbic acid, sodium ascorbate, fatty acid esters of ascorbic acid, tocopherols, alpha-tocopherol, gamma-tocopherol, delta-tocopherol, propyl gallate, octyl gallate, erythorbic acid, sodium erythorbate, dodecyl gallate, tertiary-butyl hydroquinone (TBHQ), butylated hydroxyanisole (BHA), butylated hydroxytoluene (BHT), citric acid, 4-hexylresorcinol; color retention agents; dyes or lakes; sequestrants, emulsifiers, e.g., lecithins, monoglyceride, acetylated monoglyceride, lactylated monoglyceride, sugar ester, sorbitan ester, polyglycerol ester, calcium stearoyl di laciate; stabilizers; acids, bases, and/or anticaking agents, e.g., calcium silicate, magnesium carbonate, sodium aluminosilicate.

As used herein, the terms “consumer product” and “end product” relate to end use materials that can encompass the flavor compound (i.e., compounds encompassed by Formulas (I-IV)) or flavor compositions of the presently disclosed subject matter may be used in consumer products such as foods or beverages, oral care products, animal feed and pet foods, and pharmaceuticals and over-the-counter medications. Examples of suitable consumer products include, but are not limited to carbonated fruit beverages, carbonated cola drinks, wine coolers, cordials, flavored water, powders for drinks (e.g., powdered sports or “hydrating” drinks), fruit based “smoothy” drinks, milk-based drinks, hard candy, soft candy, taffy, chocolates, sugarless candies, chewing gum, bubble gum, condiments, spices and seasonings, dry cereal, oatmeal, granola bars, condiments and preserves, soups, alcoholic beverages, energy beverages, juices, teas, coffees, salsa, gel beads, film strips for halitosis, gelatin candies, pectin candies, starch candies, lozenges, chewable tablets, breath mints, oral wash, tooth gel, cough drops, throat lozenges, throat sprays, toothpastes, mouth rinses, nicotine gums, decongestants, oral analgesics, indigestion preparations, and antacids.

The flavor compositions according to the disclosed subject matter can be in the form of a simple mixture of flavoring ingredients or in an encapsulated form, e.g., a flavoring composition entrapped into a solid matrix that can comprise wall-forming and plasticizing materials such as mono-, di-, or trisaccharides, natural or modified starches, hydrocolloids, cellulose derivatives, polyvinyl acetates, polyvinylalcohols, proteins, or pectins. Examples of particularly useful matrix materials include, for example, sucrose, glucose, lactose, levulose, fructose, maltose, ribose, dextrose, isomalt, sorbitol, mannitol, xylitol, lactitol, maltitol, pentatol, arabinose, pentose, xylose, galactose, maltodextrin, dextrin, chemically modified starch, hydrogenated starch hydrolysate, succinylated or hydrolysed starch, agar, carrageenan, gum arabic, gum accacia, tragacanth, alginates, methyl cellulose, carboxymethyl cellulose, hydroxyethyl cellulose, hydroxypropylmethyl cellulose, derivatives, gelatin, agar, alginate, and mixtures thereof. Encapsulation is well-known to persons skilled in the art, and can be performed, for instance, using techniques such as spray-drying, agglomeration or extrusion, or coating encapsulation, including coacervation and complex coacervation techniques.

In one embodiment, the compound of the presently disclosed subject matter is included/used in chewing and bubble gums and confectionaries (e.g., hard or soft candies or lozenges). Chewing gum compositions typically include one or more gum bases and other standard components such as flavoring agents, softeners, sweeteners, and the like. Flavoring agents for use in chewing gum compositions are well known and include natural flavors such as citrus oils, peppermint oil, spearmint oil, oil of wintergreen, natural menthol, cinnamon, ginger, and the like; and artificial flavors such as menthol, carvone, limonene, cinnamic aldehyde, linalool, geraniol, ethyl butyrate, and the like. As is known in the art, the ingredients used in chewing gum compositions can include sweeteners, both natural and artificial and both sugar and sugarless. Sweeteners are typically present in the chewing gum compositions in amounts of from about 20% to 80% by weight, preferably from about 30% to 60% by weight, based on the total weight of the chewing gum composition. Sugarless sweeteners include, but are not limited sugar alcohols such as Sorbitol, manifold, xylitol, hydrogenated starch hydrolysates, malitol, and the like. High intensity sweeteners such as sucralose, aspartame, neotame, salts of acesulfame, and the like, when employed, are typically present up to about 1.0% by weight.

In an alternative embodiment, the compound of the presently disclosed subject matter is included in an oral personal care product (e.g., a mouthwash, toothpaste, or toothgel). For example, a mouthwash can be prepared by dissolving a flavor composition (e.g., a flavor cocktail) (liquid or powder) that includes the compound of the presently disclosed subject matter in a solvent (e.g., water) that further includes, for example, a flavor such as menthol and a surfactant; and then mixing the resulting solution with, for example, an aqueous erythritol solution.

In one embodiment of the presently disclosed subject matter, the compound of the presently disclosed subject matter is added, directly or indirectly, to a pharmaceutical dosage form (e.g., a tablet, capsule, drop, or lozenge) that contains a therapeutically active agent (e.g., a medicament). For example, one embodiment of the presently disclosed subject matter provides a cough drop or lozenge containing the compound of the present disclosure and, optionally, further containing menthol or other medicaments for the treatment of sore throat, coughing or other upper respiratory ailments.

The presently disclosed compound can also be added to, for example, compositions for the preparation of: 1) carbonated or non-carbonated fruit beverages, carbonated cola drinks, wine coolers, fruit liquors, cordials, milk drinks, smoothy drinks, flavored water, tropical alcoholic and “virgin” drink mixes (e.g., margarita, piña colada, or “rum-runner” concentrates), and powders for drinks (e.g., powdered sports or “hydrating” drinks); 2) frozen confectioneries such as ice creams, sherbets, and ice-lollies; hard candies, soft candies, taffies, chocolates, and sugarless candies; 3) desserts such as jelly and pudding; 4) confectioneries such as cakes, cookies, chewing gums, and bubble gums; 5) condiments, spices and seasonings, dry cereals, oatmeals, and granola bars; 6) alcoholic beverages, energy beverages, juices, teas, coffees, salsa, and gel beads; 7) film strips for halitosis, and oral personal care products; 8) gelatin candies, pectin candies, starch candies, lozenges, cough drops, throat lozenges, throat sprays, and toothpastes.

The present compound can also be added to, for example; 1) Japanese confectioneries such as buns with bean-jam filling, bars of sweet jellied bean paste, and sweet jellied pounded rice; 2) jams; candies; 3) breads; 4) beverages such as green tea, oolong tea, black tea, persimmon leaf tea, Chamomile tea, Sasa veitchii tea, mulberry leaf tea, Houttuynia cordata tea, Puer tea, Mate tea, Rooibos tea, Gimunema tea, Guava tea, coffee, espresso, and hot and cold espresso and coffee products obtained by mixing espresso and/or coffee with milk, water or other liquids suitable for oral consumption (e.g., lattes, cafe au lait, cafe mocha), and cocoa; 5) soups such as Japanese flavor soup, western flavor soup, and Chinese flavor soup; 6) seasonings; 7) various instant beverages and foods; 8) various snack foods; and 9) other compositions for oral use.

### EXAMPLES

The present application is further described by means of the examples, presented below, wherein the abbreviations have the usual meaning in the art.

The use of such examples is illustrative only and in no way limits the scope and meaning of the disclosed subject matter or of any exemplified term. Likewise, the disclosed subject matter is not limited to any particular preferred embodiments described herein. Indeed, many modifications and variations of the disclosed subject matter are apparent to those skilled in the art upon reading this specification. The disclosed subject matter is therefore to be limited only by the terms of the appended claims along with the full scope of equivalents to which the claims are entitled.

The following examples are provided as specific embodiments of the present invention, wherein the abbreviations have the usual meaning in the art. The temperatures are indicated in degrees centigrade (° C.); the NMR spectral data were recorded in CDC1₃ with a 500 MHz machine for ¹H and ¹³C, the chemical displacements are indicated in ppm with respect to TMS as the standard.

### Example 1

### Synthesis of 5,9-dimethylundeca-4,8-dienal

5,9-dimethylundeca-4,8-dienal was synthesized as shown in Examples 1-A and 1-B below:

### Example 1-A

Ethyl linalool (3g), ethyl vinyl ether (23mL, 13.4 eq.), mercuric acetate (5.4g, 95 eq.) and toluene (50mL) were combined under N₂ gas and refluxed for 18 hours. Another 7.6mL (4.5 eq.) of ethyl vinyl ether and an additional 50 mL toluene were then added and reflux continued for 5 hours. The reaction was cooled, and acetic acid (1mL) was added and stirred at room temperature for 30 minutes. The mixture was extracted with diethyl ether and washed with 5% KOH, dried over MgSO₄, filtered and concentrated. The crude product was purified by column chromatography (2% ethyl acetate in hexanes) to give the product as a mixture of its isomers (2.5g; 72.2% yield). GC/MS(EI): m/z(%) 194 (M⁺, 11), 179 (1), 176 (2), 165 (14), 150 (25), 137 (36), 111 (13), 93 (52), 83 (100), 67 (55), 55 (99), 41 (99). ¹H NMR (CDCl₃): δ 9.74 (CHO). ¹³C NMR (CDC1₃): δ 202.7 and 202.6 (CHO).

### Example 1-B

Ethyl linalool (100 g), ethyl vinyl ether (103.8 g, 2.4 eq.), phenyl phosphonic acid (1.13 g, 0.01 eq.) and toluene (100 mL) were sealed in an autoclave. The vessel was purged with N₂ gas, then heated to 150° C. over 30 minutes. Then the temperature was raised to 180° C., at which point the pressure inside the vessel rose to 8 bar. After 2 hours, the reaction was complete. The product (69.6 g) was isolated as a mixture of its isomers by fractional distillation (0.25 Torr, 58° C.); 60.3% yield. GC/MS(EI): m/z(%) 194 (M⁺, 11), 179 (1), 176 (2), 165 (14), 150 (25), 137 (36), 111 (13), 93 (52), 83 (100), 67 (55), 55 (99), 41 (99). ¹H NMR (CDCl₃): δ 9.74 (CHO). ¹³C NMR (CDCl₃): δ 202.7 and 202.6 (CHO).

### Example 1-C

The compound of Formula 1 was evaluated by flavorists and perfumers.

Seven trained flavorists evaluated the compound at 1ppm in water. The flavorists determined the compound provided good mouthfeel/coat and had the following taste notes: fatty, green, aldehyde, floral, orange, citrus, herbal, leafy. Three perfumers evaluated the neat compound and determined the compound provided the following odor notes: aldehydic, floral, muguet, citrus, mandarin, mandarin rind, tangerine pith, sweet fruity, green, marine, coriander leaf.

### Example 2

### Preparation of a “Cotton Flower” Perfume Composition

A “Cotton Flower” perfume composition with a mandarin and muguet note was prepared from the compound of Formula (I) to demonstrate its use in a liquid all-purpose cleaner. The composition is provided in Table 1. The cleaner was evaluated by two Perfumers and one Evaluator.

TABLE 1

| FRAGRANCE CATEGORY | PARTS PER THOUSAND |
|---|---|
| Compound of | 2 |
| Formula (I), R = H | |
| Citrus | 290.8 |
| Floral | 304 |
| Fruity | 270 |
| Green | 8.2 |
| Musky | 21 |
| Woody | 86 |
| Solvent | 18 |
| Total | 1000 |

This composition using 2 ppt of the compound of Formula (I), adds a crisp, clean, mandarin, muguet twist to the all-purpose cleanser, with much greater volume than when the compound of Formula (I) is omitted.

### Example 3

### Preparation of a Rose Perfume Composition

A rose perfume composition was prepared from the compound of Formula Ito demonstrate its use in a bar soap. The composition is provided in Table 2. The soap was evaluated by one Perfumer and one Evaluator, and evaluated from the dry bar, during washing and also from the residue on skin after rinsing.

TABLE 2

| CATEGORY | PARTS PER THOUSAND |
|---|---|
| Compound of Formula I | 10 |
| Citrus, aldehydic, floral | |
| Animal | 1 |
| Balsamic | 1 |
| Citrus | 10 |
| Floral | 243.5 |
| Fruity | 9 |
| Gourmand | 1 |
| Green | 14 |
| Herbaceous | 1.5 |
| Marine | 1 |
| Moss | 1 |
| Musk | 355 |
| Spicey | 1 |
| Woody | 113 |
| Solvent | 142 |
| Dipropylene Glycol | 96 |
| Total | 1000 |

The fragrance with and without the compound of Formula I was dosed in the bar soap at 1.5%. This composition dosed with 10 ppt of the compound of Formula I has a nice sparkling citrus aldehydic note. It improves the fragrance with more volume and floralcy vs. when the compound of Formula I is omitted.

### Example 4

A milk & honey perfume composition was prepared from the compound of Formula Ito demonstrate its use in a shampoo. The composition is provided in Table 3. The shampoo was evaluated by one Perfumer and one Evaluator.

TABLE 3

| CATEGORY | PARTS PER THOUSAND |
|---|---|
| Compound of Formula I | 2 |
| Citrus, aldehydic, floral | |
| Aldehydic | 2 |
| Citrus | 40 |
| Floral | 386 |
| Fruity | 142 |
| Gourmand | 26 |
| Green | 2 |
| Musk | 280 |
| Powdery | 32 |
| Solvent | 88 |
| Total | 1000 |

The compound of Formula I was used at .2% in the fragrance and the fragrance was used at .8% in the shampoo base. The compound of Formula I adds a similar effect as bourgeonal to the shampoo. It cuts the sweetness of the fragrance without the compound of Formula I with a green/watery muguet floral effect.

### Example 5

An exemplary orange flavor composition comprising the compound of Formula (I).

TABLE 4

| CATEGORY | PARTS PER THOUSAND |
|---|---|
| Compound of Formula I @ 0.1% in | 0.1 |
| Triacetin Citrus | |
| Citrus | 89.2 |
| Floral/Citrus | 0.2 |
| Fruity | 10.5 |
| Total | 100 |

This composition uses 0.01% of the compound of Formula (I). The compound of Formula (I) adds a waxy freshness and natural peel note to the flavor.

The above exemplary orange flavor composition can used in an exemplary children's flavored toothpaste or tooth gel by adding the flavor composition in an amount of about 0.6% to about 1.0%, preferably 0.8%, of the total composition, to a typical silica toothpaste base or a typical silica tooth gel unflavored/unsweetened base, or a typical chalk toothpaste (PCC-calcium carbonate) base with a 1.5% hole, all known to one skilled in the art.

The above exemplary orange flavor compositon can be used in an exemplary children's fluoride mouth rinse by adding the flavor composition in an amount of about 0.05% to about 0.15%, preferably at 0.10%, of the total composition, to a typical alcohol-free mouthrinse unflavored base with a 0.5% void known to one skilled in the art.

The above exemplary orange flavor composition can also be used in an exemplary cough syrup by adding the flavor composition as liquid at about 0.2% to a typical cough syrup base known to one skilled in the art; or in an exemplary flavored anti-inflamnatory tablet by adding the flavor composition at 1% of a 20% loaded spray dried flavor to a typical acetaminophen tablet known to one skilled in the art.

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the device, method, and system of the disclosed subject matter without departing from the spirit or scope of the disclosed subject matter. Thus, it is intended that the disclosed subject matter include modifications and variations that are within the scope of the appended claims and their equivalents.

For any patents, patent applications, publications, product descriptions, and protocols are cited throughout this application, the disclosures of all of which are incorporated herein by reference in their entireties for all purposes.

1. A fragrance or flavor composition comprising a compound represented by Formula (I), 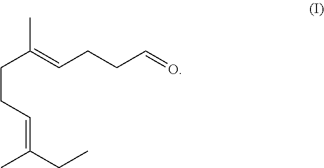
in an amount effective to impart a fragrance or flavor to the consumer product.

2. A consumer product containing the composition of claim 1.
